(19) United States
(12) Patent Application Publication
(10) Pub. No.: US 2025/0049501 A1
(43) Pub. Date: Feb. 13, 2025

(54) ASYMMETRICAL MICROWAVE ABLATION FIELD BY CURVING DIPOLE ANTENNA

(71) Applicant: Covidien LP, Mansfield, MA (US)

(72) Inventors: William J. DICKHANS, Longmont, CO (US); Scott E.M. Frushour, Boulder, CO (US); John W. Komp, Dillon, CO (US)

(73) Assignee: Covidien LP, Mansfield, MA (US)

(21) Appl. No.: 18/752,559

(22) Filed: Jun. 24, 2024

Related U.S. Application Data
(60) Provisional application No. 63/531,521, filed on Aug. 8, 2023.

(51) Int. Cl. A61B 18/18; A61B 18/00; A61M 25/01

(52) U.S. Cl. CPC A61B 18/1815; A61B 2018/00184; A61B 2018/00541; A61B 2018/00577; A61B 2018/1838; A61B 2018/1861; A61M 25/0147

(57) ABSTRACT

An articulating microwave ablation catheter and methods of use the catheter including a catheter sheathing housing therein a radiating section defining an ablation zone, a first pull wire traversing the catheter sheathing, a pull ring secured in a distal portion of the catheter sheathing and configured to receive the pull wire, and a drive mechanism operably connected to the catheter sheathing and the pull wire, wherein operation of the drive mechanism alters the length of the pull wire and a shape of a distal portion of the catheter sheathing and the location of the ablation zone defined by the radiating section.

### BACKGROUND

### Technical Field

This disclosure relates to the field of microwave ablation catheters and particularly to microwave ablation catheters capable of achieving an articulation and a user defined curvature to promote navigation and target treatment.

### Description of Related Art

There are several commonly applied medical methods, such as endoscopic procedures or minimally invasive procedures, for treating various maladies affecting organs including the liver, brain, heart, lungs, gall bladder, kidneys, and bones. Often, one or more imaging modalities, such as magnetic resonance imaging (MRI), ultrasound imaging, computed tomography (CT), or fluoroscopy are employed by clinicians to identify and navigate to areas of interest within a patient and ultimately a target for biopsy or treatment. In some procedures, pre-operative scans may be utilized for target identification and intraoperative guidance. However, real-time imaging may be required to obtain a more accurate and current image of the target area. Furthermore, real-time image data displaying the current location of a medical device with respect to the target and its surroundings may be needed to navigate the medical device to the target in a safe and accurate manner (e.g., without causing damage to other organs or tissue).

For example, an endoscopic approach has proven useful in navigating to areas of interest within a patient, and particularly so for areas within luminal networks of the body such as the lungs. To enable the endoscopic approach, and more particularly the bronchoscopic approach in the lungs, endobronchial navigation systems have been developed that use previously acquired MRI data or CT image data to generate a three-dimensional (3D) rendering, model, or volume of the particular body part such as the lungs.

The resulting volume generated from the MRI scan or CT scan may be utilized to create a navigation plan to facilitate the advancement of a navigation catheter (or other suitable medical device) through a bronchoscope and a branch of the bronchus of a patient to an area of interest. A locating or tracking system, such as an electromagnetic (EM) tracking system, may be utilized in conjunction with, for example, CT data, to facilitate guidance of the navigation catheter through the branch of the bronchus to the area of interest. In certain instances, the navigation catheter may be positioned within one of the airways of the branched luminal networks adjacent to, or within, the area of interest to provide access for one or more medical instruments, such as a microwave ablation catheter.

As will be appreciated, accurate placement of the catheter and therewith the medical instrument is important to ensure successful therapy. Improvements to the current navigation catheter systems are desired.

### SUMMARY

One aspect of the disclosure is directed to an articulating microwave ablation catheter including a catheter sheathing housing therein a radiating section defining an ablation zone; a first pull wire traversing the catheter sheathing. The catheter also includes a pull ring secured in a distal portion of the catheter sheathing and configured to receive the pull wire; and a drive mechanism operably connected to the catheter sheathing and the pull wire, where operation of the drive mechanism alters: an effective length of the pull wire, a shape of a distal portion of the catheter sheathing, and a location of the ablation zone defined by the radiating section. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The articulating microwave ablation catheter where the radiating section is a dipole antenna. The second pull wire and the first pull wire operate in concert, with one effectively lengthening and one effectively shortening, to enable articulation of the microwave ablation catheter in at least two directions. The drive mechanism is manually operated and located in a handle connected to the catheter sheathing. The catheter sheathing is configured to be received in a navigation catheter. The radiating section is housed in a distal portion of the catheter sheathing, and the distal portion of the catheter sheathing is configured to extend beyond a distal end of the navigation catheter. The pull wire is formed of graphite or carbon fiber. The drive mechanism is configured for manual operation to articulate the distal portion of the catheter sheathing. The drive mechanism is configured for robotic operation to articulate the distal portion of the catheter sheathing. Upon articulation of the microwave ablation catheter, the ablation zone of the radiating sections moves between 1 and 3 mm in a direction of the articulation. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A further aspect of the disclosure is directed to a method including receiving an indication that a microwave ablation catheter has been navigated to location within the lungs of a patient; and articulating the microwave ablation catheter, where articulation adjusts an ablation zone of a radiating section of the microwave ablation catheter between 1 and 3 mm in a direction of the articulation. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The method where the articulation ensures that a therapy margin is achieved around a lesion. The method further including planning a navigation pathway through the lungs of the patient. The navigation pathway is planned from pre-procedure images. The method further including planning an articulation of the microwave ablation catheter. The method further including acquiring images to confirm placement of the microwave ablation catheter relative to a lesion in the patient. The method further including acquiring images to assess a completeness of an ablation. The method further including straightening or articulating the microwave ablation catheter in a second direction to alter the ablation zone to a second portion of the lesion. The method further including imaging the lesion to confirm a margin around the lesion. The method further including determining whether more lesions are identified in a pathway plan, receiving an indication that the microwave ablation catheter is at a location of a second lesion, articulating the microwave ablation catheter, and ablating the second lesion. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

### BRIEF DESCRIPTION OF THE DRAWINGS

Various aspects and embodiments of the disclosure are described hereinbelow with references to the drawings, wherein:

FIG. 1 is a schematic view of a navigation system in accordance with the disclosure;

FIG. 2 is a perspective view of a distal end of a microwave ablation catheter in proximity to a lesion in accordance with the disclosure;

FIGS. 3A and 3B are perspective views of a catheter drive mechanism in accordance with the disclosure;

FIG. 4 is a flow chart of a method in accordance with the disclosure;

FIG. 5 is a side view of a distal portion of a microwave ablation catheter in accordance with the disclosure; and

FIG. 6 is a schematic view of a computing system in accordance with the disclosure.

### DETAILED DESCRIPTION

The disclosure is directed to an articulating microwave ablation catheter for use in applying therapy to targets (e.g., lesions and tumors) in or near navigable luminal networks such as the airways of the lungs. Articulation allows for a user to alter the ablation zone formed by the application of the microwave energy to create an asymmetric ablation zone. This asymmetric ablation zone is created by the curving of the antenna. By bending the shape of the microwave ablation catheter, a margin (treated tissue beyond the tumor or lesion) can be shifted by a measurable amount (e.g., 2-3 mm). While this shift may be considered relatively small, when the total desired margin is, for example, 5 mm, even this shift represents a significant change in the efficacy of microwave ablation catheters used in, for example, lung tumor therapy. The ability to shift the position of the margin and create an asymmetric ablation zone through articulation of the distal portion of the microwave ablation catheter improves the ability of the clinician to achieve a successful treatment and reduces the required accuracy of placement to achieve that successful treatment.

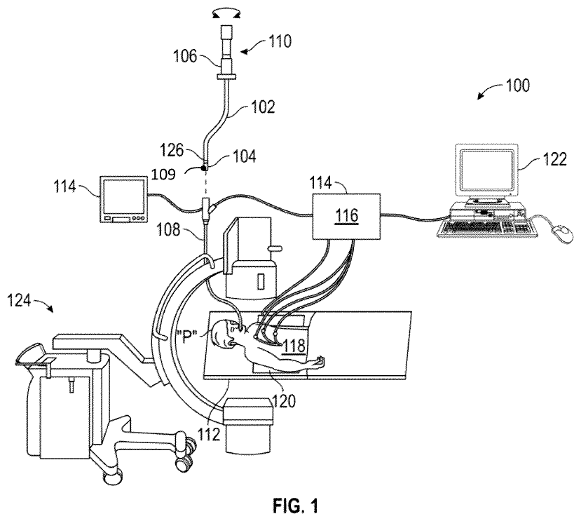

FIG. 1 is a perspective view of an exemplary system for facilitating navigation of a catheter to a soft tissue target via airways of the lungs. As shown in FIG. 1, catheter 102 is part of a catheter guide assembly 106. In one embodiment, catheter 102 is inserted into a bronchoscope 108 for access to a luminal network of the patient P. Specifically, catheter 102 of catheter guide assembly 106 may be inserted into a working channel of bronchoscope 108 for navigation through a patient's luminal network. The catheter 102 may itself include imaging capabilities via an integrated camera or optics component 109 and a separate bronchoscope 108 is not strictly required. A locatable guide (LG) 110 (a second catheter), including a sensor 104 may be inserted into catheter 102 and locked into position such that sensor 104 extends a desired distance beyond the distal tip of catheter 102. The position and orientation of sensor 104 relative to a reference coordinate system, and thus the distal portion of catheter 102, within an electromagnetic field can be derived. Catheter guide assemblies 106 are currently marketed and sold by Medtronic PLC under the brand names SUPERDIMENSION® Procedure Kits, ILLUMISITE™ Endobronchial Procedure Kit, ILLUMISITE™ Navigation Catheters, or EDGE™ Procedure Kits, and are contemplated as useable with the disclosure.

System 100 generally includes an operating table 112 configured to support a patient P and monitoring equipment 114 coupled to bronchoscope 108 or catheter 102 (e.g., a video display, for displaying the video images received from the video imaging system of bronchoscope 108 or the catheter 102); a locating or tracking system 115 including a locating module 116, a plurality of reference sensors 18 and a transmitter mat 120 including a plurality of incorporated markers; and a computing device 122 including software and/or hardware used to facilitate identification of a target, pathway planning to the target, navigation of a medical device to the target, and/or confirmation and/or determination of placement of catheter 102, or a suitable device therethrough, relative to the target.

As is typical of catheter guide assembly 10 navigation the six degrees-of-freedom electromagnetic locating or tracking system 115, or other suitable system for determining position and orientation of a distal portion of the catheter 102, is utilized, as will be outlined below for performing registration of a detected position of the sensor 104 and a 3D model generated from an images received from an imaging modality (e.g., Computed Tomography (CT), Cone Beam Computed Tomography (CBCT), Magnetic Resonance Imaging (MRI) and others). Tracking system 114 includes the tracking module 116, a plurality of reference sensors 118, and the transmitter mat 120 (including the markers). Tracking system 114 is configured for use with a locatable guide 110 and particularly sensor 104. As described above, locatable guide 110 and sensor 104 are configured for insertion through catheter 102 into patient P's airways (either with or without bronchoscope 108) and are selectively lockable relative to one another via a locking mechanism.

Transmitter mat 120 is positioned beneath patient P. Transmitter mat 120 generates an electromagnetic field around at least a portion of the patient P within which the position of a plurality of reference sensors 118 and the sensor 104 can be determined with use of a tracking module 116. A second electromagnetic sensor 126 may also be incorporated into the end of the catheter 102. The second electromagnetic sensor 126 may be a five degree-of-freedom sensor or a six degree-of-freedom sensor. One or more of reference sensors 118 are attached to the chest of the patient P. Registration is generally performed to coordinate locations of the three-dimensional model and two-dimensional images from the planning phase, with the patient P's airways as observed through the bronchoscope 108 and allow for the navigation phase to be undertaken with knowledge of the location of the sensor 104.

Registration of the patient P's location on the transmitter mat 120 may be performed by moving sensor 104 through the airways of the patient P. More specifically, data pertaining to locations of sensor 104, while locatable guide 110 is moving through the airways, is recorded using transmitter mat 120, reference sensors 118, and tracking system 114. A shape resulting from this location data is compared to an interior geometry of passages of a 3D model, and a location correlation between the shape and the 3D model based on the comparison is determined, e.g., utilizing the software on computing device 122. In addition, the software identifies non-tissue space (e.g., air filled cavities) in the three-dimensional model. The software aligns, or registers, an image representing a location of sensor 104 with the three-dimensional model and/or two-dimensional images generated from the three-dimension model, which are based on the recorded location data and an assumption that locatable guide 110 remains located in non-tissue space in patient P's airways. Alternatively, a manual registration technique may be employed by navigating the bronchoscope 108 with the sensor 104 to pre-specified locations in the lungs of the patient P, and manually correlating the images from the bronchoscope to the model data of the three-dimensional model.

Though described herein with respect to EMN systems using EM sensors, the instant disclosure is not so limited and may be used in conjunction with flexible sensors such as fiber-bragg grating sensors, inertial measurement unit (IMU), ultrasonic sensors, or without sensors. Any of these sensors may be employed as sensor 104 or sensor 126, without departing from the scope of the disclosure. Additionally, as outlined below the methods described herein may be used in conjunction with robotic systems such that robotic actuators drive the catheter 102 or bronchoscope 108 proximate the target.

In accordance with aspects of the disclosure, the visualization of intra-body navigation of a medical device (e.g., a biopsy tool or a therapy tool), towards a target (e.g., a lesion) may be a portion of a larger workflow of a navigation system. An imaging device 124 (e.g., a CT imaging device such as a cone-beam computed tomography (CBCT) device, including but not limited to Medtronic plc's O-arm™ system) capable of acquiring 2D and 3D images or video of the patient P is also included in this particular aspect of system 100. The images, sequence of images, or video captured by imaging device 124 may be stored within the imaging device 124 or transmitted to computing device 122 for storage, processing, and display. Additionally, imaging device 124 may move relative to the patient P so that images may be acquired from different angles or perspectives relative to patient P to create a sequence of images, such as a fluoroscopic video. The pose of imaging device 124 relative to patient P while capturing the images may be estimated via markers incorporated with the transmitter mat 120. The markers are positioned under patient P, between patient P and operating table 112 and between patient P and a radiation source or a sensing unit of imaging device 124. The markers incorporated with the transmitter mat 120 may be two separate elements which may be coupled in a fixed manner or alternatively may be manufactured as a single unit. Imaging device 124 may include a single imaging device or more than one imaging device.

Computing device 122 may be any suitable computing device including a processor and storage medium, wherein the processor is capable of executing instructions stored on the storage medium. Computing device 122 may further include a database configured to store patient data, CT data sets including CT images, fluoroscopic data sets including images and video, 3D reconstruction, navigation plans, and any other such data. Although not explicitly illustrated, computing device 122 may include inputs, or may otherwise be configured to receive, CT data sets, fluoroscopic images/video and other data described herein. Additionally, computing device 122 includes a display configured to display graphical user interfaces. Computing device 122 may be connected to one or more networks through which one or more databases may be accessed.

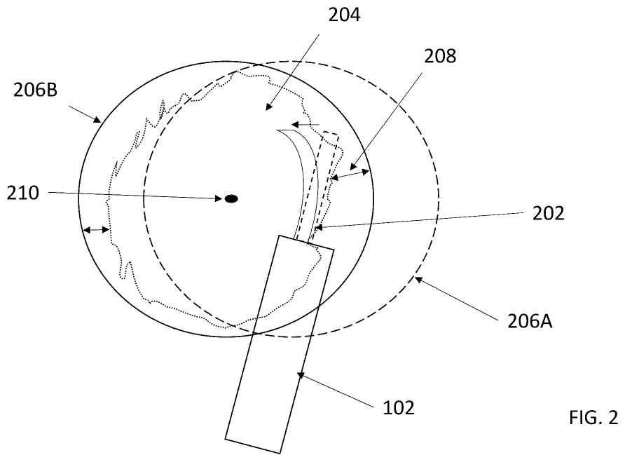

FIG. 2 depicts an aspect of the disclosure in which a microwave ablation catheter 202 has been inserted into the catheter 102 (e.g., following removal of the LG 110) following placement within a tumor or lesion 204. The microwave ablation catheter 202 may include a dipole construction, examples of which are described in commonly assigned U.S. Pat. No. 9,119,650 entitled, MICROWAVE ENERGY-DELIVERY DEVICE AND SYSTEM, issued Sep. 1, 2015, and U.S. Pat. No. 10,813,691 entitled MINIATURIZED MICROWAVE ABLATION ASSEMBLY, issued Oct. 27, 2020, the entire contents of which are both incorporated herein by reference.

In accordance with the disclosure and the dipole construction, the microwave ablation catheter 202 generally produced a spherical ablation zone 206A when in an unarticulated or straight configuration. By selecting a power and duration of energy application a margin 208 is established around the tumor or lesion 204 during treatment. However, to ensure that the margin extends around the entire lesion or tumor 204, it is desirable to place the microwave ablation catheter on or near the center 210 of the lesion or tumor 204. As can be seen in FIG. 2, the microwave ablation catheter 202 is not located at the center of the tumor or lesion 204.

Placement in the center of the tumor or lesion 204 can be very difficult. Initially, the tissues of the lungs, particularly as the periphery is approached, is very flexible and is distorted even by the movement of the catheter 102 and the microwave ablation catheter 202. Further, in some instances, it is desirable to move the microwave ablation catheter 202 away from certain structures (e.g., the Hilum) to prevent or limit damage to these structures by application of energy via the microwave ablation catheter 202 to the tissue. Further, while EMN navigation systems are quite good and improvements have been developed to ensure accurate placement at the tumor or lesion 204, because of the tortuous nature, airways tools, such as the microwave ablation catheter 202, may not have the ability to reach the center 210 of the tumor or lesion 204.

To address the challenges in the precise placement of the microwave ablation catheter 202, the microwave ablation catheter is articulatable in at least one direction (as shown by the arrow in FIG. 2). In accordance with the disclosure, by articulating and therefore altering the shape of the microwave ablation catheter 202, the ablation zone shifts to 206B and helps to ensure that the margin 208 is achieved on all sides of the lesion or tumor 204.

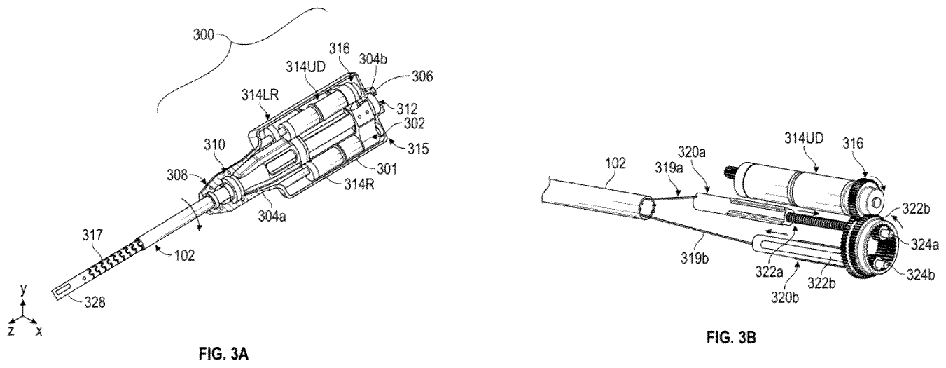

In accordance with the disclosure, the catheter 102 and its articulation and orientation relative to a target is achieved using a drive mechanism 300 which may be handheld or incorporated into a robotic system. One example of such a drive mechanism can be seen in FIG. 3A which depicts a housing including three drive motors to manipulate a catheter extending therefrom in 5 degrees of freedom (e.g., left, right, up, down, and rotation). Other types of drive mechanisms including fewer or more degrees of freedom and other manipulation techniques may be employed without departing from the scope of the disclosure.

As noted above, FIG. 3A depicts a drive mechanism 300 housed in a body 301 and mounted on a bracket 302 which integrally connects to the body 301. The catheter 102 connects to and in one embodiment forms an integrated unit with internal casings 304a and 304b and connects to a spur gear 306. This integrated unit is, in one embodiment, rotatable in relation to the housing 301, such that the catheter 102, internal casings 304 a-b, and spur gear 306 can rotate about shaft axis “z”. The catheter 102 and integrated internal casings 304 a-b are supported radially by bearings 308, 310, and 312. Though drive mechanism 300 is described in detail here, other drive mechanisms may be employed to enable a robot or a clinician to drive the catheter to a desired location without departing from the scope of the disclosure.

An electric motor 314R, may include an encoder for converting mechanical motion into electrical signals and providing feedback to the computing device 122. Further, the electric motor 314R (R indicates this motor if for inducing rotation of the catheter 102) may include an optional gear box for increasing or reducing the rotational speed of an attached spur gear 315 mounted on a shaft driven by the electric motor 314R. Electric motors 314LR (LR referring to left-right movement of an articulating portion 317 of the catheter 102) and 314UD (referring to up-down movement of the articulating portion 317), each motor optionally includes an encoder and a gearbox. Respective spur gears 316 and 318 drive up-down and left-right steering cables, as will be described in greater detail below. All three electric motors 314 R, LR, and UD are securely attached to the stationary frame 302, to prevent their rotation and enable the spur gears 315, 316, and 318 to be driven by the electric motors.

FIG. 3B depicts details of the drive mechanism 300 causing articulating portion 317 of catheter 102 to articulate. Specifically, the following depicts the manner in which the up-down articulation is contemplated in one aspect of the disclosure. Such a system alone, coupled with the electric motor 314UD for driving the spur gear 1216 would accomplish articulation as described above in a two-wire system. However, where a four-wire system is contemplated, a second system identical to that described immediately hereafter, can be employed to drive the left-right cables. Accordingly, for ease of understanding just one of the systems is described herein, with the understanding that one of skill in the art would readily understand how to employ a second such system in a four-wire system. Those of skill in the art will recognize that other mechanisms can be employed to enable the articulation of a distal portion of a catheter and other articulating catheters may be employed without departing from the scope of the disclosure.

To accomplish up-down articulation of the articulating portion 317 of the catheter 102, pull wires 319 a-b may be employed. The distal ends of the pull wires 319 a-b are attached to, or at, or near the distal end of the catheter 102. The proximal ends of the pull wires 319 a-b are attached to the distal tips of the posts 320 a, and 320 b. As shown in FIG. 3B, the posts 320a and 320b reciprocate longitudinally, and in opposing directions. Movement of the posts 320a causes one pull wire 319pull wire 319 a to effectively lengthen and at the same time, opposing longitudinal movement of post 320 b causes pull wire 319 b to effectively shorten. The combined effect of the change in effective length of the pull wires 319 a-b is to cause the articulating portion 317 of catheter 102 shaft to be compressed on the side in which the pull wire 319 b is shortened, and to elongate on the side in which pull wire 319pull wire 319 a is effectively lengthened.

The opposing posts 320a and 320b have internal left-handed and right-handed threads, respectively, at least at their proximal ends. As shown in FIG. 3A housed within casing 304 b are two threaded shafts 322a and 322b, one is left-hand threaded and one right-hand threaded, to correspond and mate with posts 320a and 320b. The shafts 322a and 322b have distal ends which thread into the interior of posts 320a and 320b and proximal ends with spur gears 324a and 324b. The shafts 322a and 322b have freedom to rotate about their axes. The spur gears 324a and 324b engage the internal teeth of planetary gear 326. The planetary gear 326 also has an external tooth which engages the teeth of spur gear 318 on the proximal end of electric motor 314UD.

To articulate the catheter in the upwards direction, a clinician may activate via an activation switch (not shown) for the electric motor 314UD causing it to rotate the spur gear 318, which in turn drives the planetary gear 326. The planetary gear 326 is connected through the internal gears 324a and 324b to the shafts 322a and 322b. The planetary gear 326 will cause the gears 324a and 324b to rotate in the same direction. The shafts 322a and 322b are threaded, and their rotation is transferred by mating threads formed on the inside of posts 320a and 320b into linear motion of the posts 320a and 320b. However, because the internal threads of post 320 a are opposite that of post 320b, one post will travel distally and one will travel proximally (i.e., in opposite directions) upon rotation of the planetary gear 326. Thus, the upper pull wire 319 a is pulled proximally to lift the catheter 102, while the lower pull wire 319 b must be relaxed. The actual lengths of the pull wires 319 does not change but their effective lengths as the posts 320 are driven onto or off of the shafts 322. As stated above, this same system can be used to control left-right movement of the end effector, using the electric motor 314LR, its spur gear 316, a second planetary gear (not shown), and a second set of threaded shafts 322 and posts 320 and two more pull wires 319. Moreover, by acting in unison, a system employing four steering cables can approximate the movements of the human wrist by having the three electric motors 314 and their associated gearing and pull wires 319 computer controlled by the computing device 122.

In accordance with one aspect of the disclosure, as the catheter 102 is advanced into the luminal network of a patient (e.g., the airways of the lungs), an application on the computing device, can receive inputs from the camera 109, sensor 104 or sensor 126 and direct the electric motors 314 to articulate or rotate the catheter 102 to be advanced along a path to a target within the patient. In the catheter assembly 106 may be handheld by the clinician and as the clinician advances the catheter 102 into the patient, the application makes the determination of articulations of the end of the catheter 102 required to allow the catheter 102 to reach a target location. Further, the drive mechanism 300 may be incorporated into one or more robotic arms or a sled (not shown) for movement of the catheter 102 and drive mechanism 300 in the z-direction (along the longitudinal axis of the catheter 102).

The drive mechanism 300 may receive inputs from computing device 122 or another mechanism through which the surgeon specifies the desired action of the catheter 102. Where the clinician controls the movement of the catheter 102, this control may be enabled by a directional button, a joystick such as a thumb operated joystick, a toggle, a pressure sensor, a switch, a trackball, a dial, an optical sensor, and any combination thereof. The computing device responds to the user commands by sending control signals to the motors 314. The encoders of the motors 314 provide feedback to the control unit 24 about the current status of the motors 314.

In accordance with the disclosure, and as outlined in greater detail below, the drive mechanism 300 receives signals derived by the computing device 122 to drive the catheter 102 (e.g., extend and retract pull-wires) to maintain the orientation of the distal tip of the catheter 102 despite extension of a tool such as a biopsy needle or ablation catheter or movements caused by respiration and cardiac cycles.

As described in connection with FIGS. 3A and 3B, catheter 102 is operated on its proximal end through a collection of controls for rotation and distal tip deflection. In contrast, to the embodiment described in connection with FIGS. 3A and 3B, a manually advanced catheter 102 may not include the motor 314R, relying instead on manual manipulation for rotation of the catheter 102. Alternatively, the drive mechanism may include only a single wire 319, or a single pair of wires 319 a, 319 b. In such an embodiment, articulation is enabled in a single or in a pair of wires in opposite directions. One or more knobs or levers or wheels on the proximal handle control or energize the energize the respective motor 314 to enable for distal tip articulation. Rotation and advancement/extraction of the catheter 102 are controlled directly by the user's hand pushing, pulling, and rotating the catheter 102 within the patient. As described in connection with FIGS. 3A and 3B, any or all of these manual controls can be removed, and users indirectly control the catheter operation through an interface to the motors such as a joystick. Navigation may also be fully automatic with user oversight.

While FIGS. 3A and 3B are initially directed at the articulation drive 300 of catheter 102, a similar system may be employed on a proximal end of the microwave ablation catheter 202. For example, a handle (not shown) on a proximal end of the microwave ablation catheter 202 may house the arrangement depicted in FIG. 3B. Essentially rather than be connected to catheter 102, the arrangement of FIG. 3B is connected to microwave ablation catheter 202 and the pair of wires 319a and 319b are received in channels formed in the microwave ablation catheter 202 and may connect to a pull ring formed proximate the distal portion of the microwave ablation catheter 202 to effectuate a change in shape or articulation as depicted in FIG. 2.

FIG. 3B depicts the use of two pull wires 319a and 319b, however, in some embodiments a single pull wire 319 may be employed. The single pull wire 319 enables articulation in just one direction, however, to articulate in any other direction, the microwave ablation catheter 202 may be rotated to the desired plane in which the microwave ablation catheter 202 is to articulate. Further, though described herein as being motorized, those of skill in the art will recognize that the articulation of the microwave ablation catheter 202 may be manual, for example, in a similar fashion to the articulation of endoscopes and bronchoscopes using a manually operated lever on the body of the endoscope.

In accordance with the disclosure, the pull wire 319 articulates a distal portion of the microwave ablation catheter 202 as depicted in FIG. 2. This articulation causes the transmitting antenna portion (e.g., a dipole) of the microwave ablation catheter 202, to bend. This bending forms an asymmetric ablation zone 206B, which shifts in the direction of the articulation. As noted above, the articulation allows for the effective margin 208 around the tumor or lesion 204 to be shifted in the direction of the articulation. As noted above, this articulation compensates for any errors in or limits to navigation, such as those caused by the physiology of the patient, to achieve an effective ablation. However, as will be appreciated, there are additional benefits. Initially, the microwave ablation catheter 202, after articulation in a first direction, can be straightened or articulated in a second direction to create a larger ablation zone 206B and to accommodate tumors or lesions 204 having a large dimension or an odd shape. This may be repeated as necessary to ensure the margin 208 is achieved around the tumor or lesion 204.

Further, through the use of the articulation, the tissue of the tumor or lesion 204 may be moved slightly. This movement may enhance the efficacy of the microwave energy applied via the microwave ablation catheter 202, and further may move the ablation zone 206 away from certain tissues to limit damage to those tissues. In particular, movement of the tumor may create space between the tumor and a heat sink such as a large blood vessel thus increasing the efficient application of energy to the tumor or lesion 204, and prevent damage to the blood vessel. Alternatively, the articulation generated by the pull wires 319 may overcome resistance to movement from the tissue to bring the ablation zone 206 to the tumor or lesion 204.

As will be appreciated, because of the use of microwave energy, the pull wires 319 may be formed of a material with a high melt point such as glass, graphite, or carbon fiber. Because these materials are also non-conducting, they avoid interaction with the antenna (e.g., dipole) of the microwave ablation catheter 202, and thus do not cause undesirable heating within the microwave ablation catheter 202.

In yet a further aspect of the disclosure, rather than using articulation mechanisms of 3A and 3B, the pull wires 319 may employ one or more shape memory metal alloys, such as Nitinol. As is understood, shape memory alloy wires change shape as they are heated. In accordance with the disclosure, once the microwave ablation catheter 202 is placed near the tumor or lesion 204, microwave energy which is emitted from the antenna heats the tissue of the tumor or lesion 204. In addition to heating the tumor or lesion 204, the pull wire 319 of shape memory alloy also begins to change shape. Shape memory wires can be “trained” such that their change in shape caused by heating is set prior to incorporation into the microwave ablation catheter 202. As such, during the heating the ablation zone shifts from 206A to 206B, to ensure that the margin 208 is achieved around the tumor or lesion 204. In some aspects, the energy applied to the antenna of the microwave ablation catheter 202 may be dithered (switched on and off) such that a temperature of the shape memory metal alloy is maintained within a range that achieves a desired amount of articulation.

In accordance with the disclosure, in addition to the pathway planning, described above, identifying the pathway to arrive at a tumor or lesion 204, a curvature or amount of articulation needed to achieve a desired margin 208 around a tumor or lesion 204 that is identified in the pre-procedure images can also be pre-planned. In this way locations for the potential placement of the catheter 102 and further placement of the microwave ablation catheter 202 can be assessed such that the easiest pathway for navigation to the tumor or lesion 204, in combination with a level of articulation is identified and selected for navigation. In some aspects of the disclosure, whether manually advanced or robotically driven, the catheter102 and therewith the microwave ablation catheter 202 can be advanced along the navigation pathway and the articulation of the microwave ablation catheter 202 driven based on the pre-planned location and articulation magnitude. This planning may also take into account possible movement of the tumor or lesion 204 caused by the articulation when acting on the tissue around the tumor or lesion in vivo. This movement may be managed and movement of the tumor or lesion 204 in the pathway plan can be shown to move with the articulation.

Still further, following navigation to the tumor or lesion 204, one or more imaging devices 124 may be used to confirm the placement of the microwave ablation catheter 202 near the tumor or lesion 204. The images may be in 2D fluoroscopy images, 3D reconstructions from fluoroscopy, CT, or CBCT images and 3D models formed therefrom. With the imaging depicting the tumor or lesion 204 and the positioning of the microwave ablation catheter 202 following navigation and articulation. An indicator on a user interface may depict the ablation zone 206B relative to the tumor or lesion 204. This ablation zone 206 may be altered by inputting a duration or power level to be applied by the microwave ablation catheter 202 such that the margin 208 is achieved.

In accordance with another aspect of the disclosure, the imaging device 124 may be used either periodically during or following an application of microwave ablation energy via the microwave ablation catheter 202 to assess the progression of the ablation. As will be appreciated, as the tissue denatures as a result of the application of energy the image of those tissues changes, by assessing the progression of the ablation through the capture of images, data is acquired that may be analyzed by artificial intelligence, machine learning, neural networks and the like. These assessments may provide guidance to a clinician on proper placement and articulation of microwave ablation catheter 202 to achieve the desired therapy. This data may be employed by an application running on computing device 122 to provide input to the navigation and articulation planning described herein.

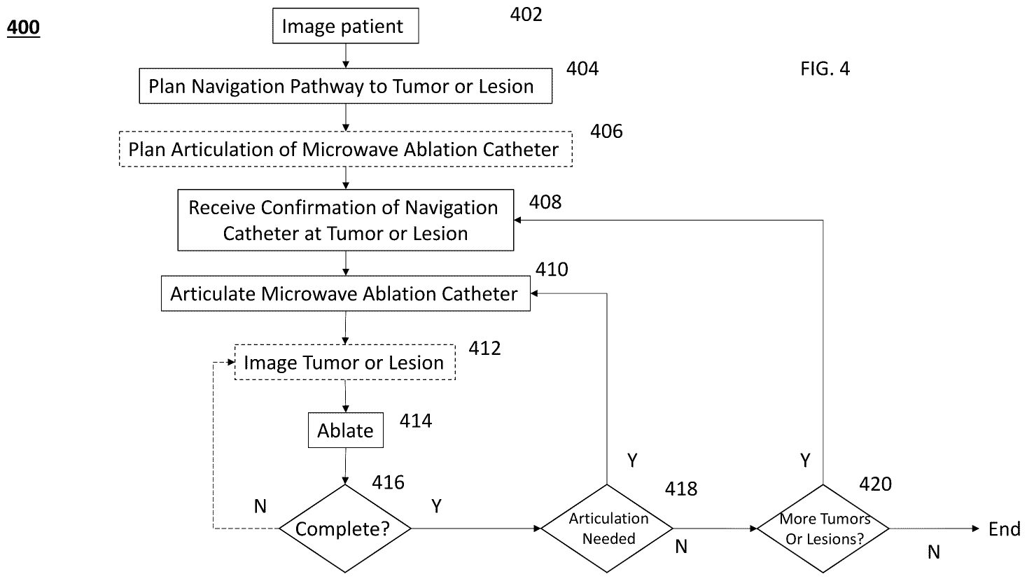

FIG. 4 depicts a method 400 in accordance with the disclosure. At step 402 images of the patient are acquired. For example, these may be pre-procedure CT images as described elsewhere herein, however, the acquisition of images is not so limited and the images may be acquired intra-procedurally via bronchoscope, fluoroscope, or intraprocedural CBCT imaging modalities, among others. At step 404 the images either pre-procedure images or intra-procedural images are analyzed, a tumor or lesion identified, and a pathway to the tumor or lesion through the airways of the patient is planned. Optionally at step 406 the pathway plan may also contemplate and plan for the articulation necessary for the microwave ablation catheter 202 for placement near the tumor or lesion 204. Whether step 406 is undertaken or not the navigation of patient is commenced and following the pathway plan, the catheter 102 is navigated to a position proximate the tumor or lesion 204. A sensor 104, 126 outputs a signal that the navigation application running on computing device 122 utilizes to indicate the progress of the catheter 102 through the patient. Eventually, the sensor 104,126 provides an output indicating that the catheter 102 has arrived at the location of the tumor or lesion 204 at step 408. Typically following navigation of the catheter 102 to a location near the tumor or lesion 204, the microwave ablation catheter 202 is inserted into the catheter 102 and extended some distance for a distal end of the catheter 102. At step 410 the microwave ablation catheter 202 is articulated, as shown in FIG. 2 such that the distal portion of the microwave ablation catheter 202 curves towards the tumor or lesion 204. At step 412, the microwave ablation catheter 202 and its relative placement to the tumor or lesion 204 may be optionally imaged to confirm placement of the microwave ablation catheter 202 relative to the tumor or lesion. At step 414 ablation is commenced. At step 416, an inquiry is made whether the ablation is complete. If not, the method returns to step 412 for optional imaging and then continued ablation at step 414. If the response to the inquiry at step 416 is yes, then the method proceeds to an inquiry at step 418 to determine whether articulation is required, for example for a large tumor or an irregular shaped tumor. If the response to the inquiry is yes, the method returns to step 410 where steps 410 through 416 are repeated as needed. If the response to the inquiry at step 418 is no, the method proceeds to step 420 for a determination of whether there are more tumors or lesions in need of therapy at step 420. If the response to the inquiry at step 420 is yes, the method returns to step 408 and the catheter 102 is navigated following the pathway plan to be navigated to the next tumor or lesion 204 until confirmation that the catheter 102 is proximate the tumor or lesion are received, and steps 410-418 are repeated as necessary. If the response to the inquiry at step 420 is no, then the method ends.

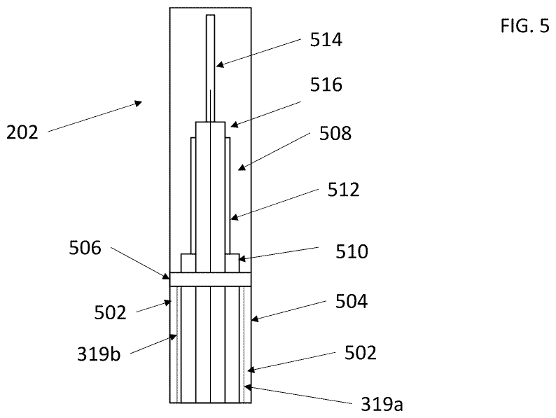

FIG. 5 depicts a cross-sectional view of a distal portion of a microwave ablation catheter 202 in accordance with the disclosure. Though depicted is a dipole antenna, the disclosure is not so limited and a monopole antenna may be employed without departing from the scope of the disclosure. Pull wires 319a and 319b traverse the microwave ablation catheter 202 in channels 502 formed in a sheathing 504 of the microwave ablation catheter 202 and end in a pull ring 506. A radiating section 508 of a microwave ablation antenna is depicted here distal of the pull ring 506. In this instance the radiating section is a dipole antenna with a balun 510. A proximal radiating section 512 is a first pole of the dipole and a distal radiating section 514 is a second pole of the dipole, the proximal radiating section 512 and the distal radiating section 514 are separated by a feed gap 516. The microwave ablation catheter 202 may be water jacketed with water flowing through the microwave ablation catheter 202 and around at least the radiating section 508. Other orientations of microwave ablation antenna 202 including non-water jacketed, different construction of radiating sections, and placement of the pull wire 319a and 319b are contemplated within the scope of the disclosure. While the above disclosures have been directed to articulating microwave ablation catheter 202, the disclosure is not so limited. In a further aspect of the disclosure, rather than the microwave ablation catheter 202 being articulated, the catheter 102 employes an articulation mechanism similar to that described in connection with FIGS. 3A, 3B, and 5. Further, rather than the microwave ablation catheter 202 being articulatable, the microwave ablation catheter 202 is flexible and has a pre-formed curvature. The flexibility of the microwave ablation catheter 202 causes the microwave ablation catheter to straighten when within the catheter 102. Once advanced from the articulating catheter 102 the pre-formed curvature returns to the distal portion of the microwave ablation catheter 202, and a similar change in orientation of the microwave ablation catheter 202 relative to the catheter 102 as depicted in FIG. 2 can be achieved.

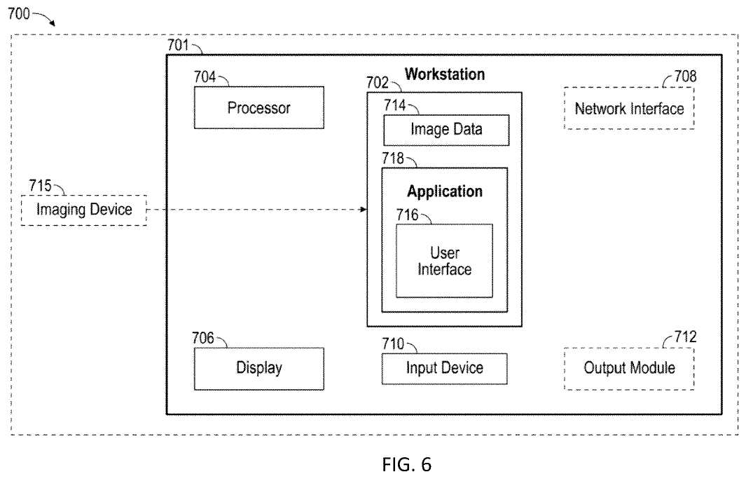

Reference is now made to FIG. 6, which is a schematic diagram of a system 700 configured for use with the methods of the disclosure including the method of FIG. 4. System 700 may include a workstation 701, and optionally an imaging device 715 (e.g., a fluoroscope or an ultrasound device). In some embodiments, workstation 701 may be coupled with imaging device 715, directly or indirectly, e.g., by wireless communication. Workstation 701 may include a memory 702, a processor 704, a display 706 and an input device 710. Processor or hardware processor 704 may include one or more hardware processors. Workstation 701 may optionally include an output module 712 and a network interface 708. Memory 702 may store an application 718 and image data 77. Application 718 may include instructions executable by processor 704 for executing the methods of the disclosure including the method of FIG. 4.Application 718 may further include a user interface 716. Image data 714 may include the CT scans, the generated fluoroscopic 3D reconstructions of the target area and/or any other fluoroscopic image data and/or the generated one or more slices of the 3D reconstruction. Processor 704 may be coupled with memory 702, display 706, input device 710, output module 712, network interface 708 and imaging device 715. Workstation 701 may be a stationary computing device, such as a personal computer, or a portable computing device such as a tablet computer. Workstation 701 may embed a plurality of computer devices.

Memory 702 may include any non-transitory computer-readable storage media for storing data and/or software including instructions that are executable by processor 704 and which control the operation of workstation 701 and, in some embodiments, may also control the operation of imaging device 715. Imaging device 715 may be used to capture a sequence of fluoroscopic images based on which the fluoroscopic 3D reconstruction is generated and to capture a live 2D fluoroscopic view according to this disclosure. In an embodiment, memory 702 may include one or more storage devices such as solid-state storage devices, e.g., flash memory chips. Alternatively, or in addition to the one or more solid-state storage devices, memory 702 may include one or more mass storage devices connected to the processor 704 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 704. That is, computer readable storage media may include non-transitory, volatile, and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by workstation 1001.

Application 718 may, when executed by processor 704, cause display 706 to present user interface 716. User interface 716 may be configured to present to the user a single screen including a three-dimensional (3D) view of a 3D model of a target from the perspective of a tip of a medical device, a live two-dimensional (2D) fluoroscopic view showing the medical device, and a target mark, which corresponds to the 3D model of the target, overlaid on the live 2D fluoroscopic view. User interface 716 may be further configured to display the target mark in different colors depending on whether the medical device tip is aligned with the target in three dimensions.

Network interface 708 may be configured to connect to a network such as a local area network (LAN) consisting of a wired network and/or a wireless network, a wide area network (WAN), a wireless mobile network, a Bluetooth network, and/or the Internet. Network interface 708 may be used to connect between workstation 701 and imaging device 715. Network interface 708 may also be used to receive image data 714. Input device 710 may be any device by which a user may interact with workstation 701, such as, for example, a mouse, keyboard, foot pedal, touch screen, and/or voice interface. Output module 712 may include any connectivity port or bus, such as, for example, parallel ports, serial ports, universal serial busses (USB), or any other similar connectivity port known to those skilled in the art. From the foregoing and with reference to the various figures, those skilled in the art will appreciate that certain modifications can be made to the disclosure without departing from the scope of the disclosure.

### Examples

Aspects of this disclosure are described in greater detail in connection with the following numbered paragraphs, in which:

Example 1—An articulating microwave ablation catheter comprising: a catheter sheathing housing therein a radiating section defining an ablation zone; a first pull wire traversing the catheter sheathing; a pull ring secured in a distal portion of the catheter sheathing and configured to receive the pull wire; and a drive mechanism operably connected to the catheter sheathing and the pull wire, wherein operation of the drive mechanism alters: an effective length of the pull wire, a shape of a distal portion of the catheter sheathing, and a location of the ablation zone defined by the radiating section.

Example 2—The articulating microwave ablation catheter of Example 1, wherein the radiating section is a dipole antenna.

Example 3—The articulating microwave ablation catheter of one of the preceding examples, further comprising a second pull wire, wherein the second pull wire and the first pull wire operate in concert, with one effectively lengthening and one effectively shortening, to enable articulation of the microwave ablation catheter in at least two directions.

Example 4—The articulating microwave ablation catheter of one of the preceding examples, wherein the drive mechanism is manually operated and located in a handle connected to the catheter sheathing.

Example 5—The articulating microwave ablation catheter of one of the preceding examples, wherein the catheter sheathing is configured to be received in a navigation catheter.

Example 6—The articulating microwave ablation catheter of example 5, wherein the radiating section is housed in a distal portion of the catheter sheathing, and the distal portion of the catheter sheathing is configured to extend beyond a distal end of the navigation catheter.

Example 7—The articulating microwave ablation catheter of one of the preceding examples, wherein the pull wire is formed of graphite or carbon fiber.

Example 8—The articulating microwave ablation catheter of one of the preceding examples, wherein the drive mechanism is configured for manual operation to articulate the distal portion of the catheter sheathing; or wherein the drive mechanism is configured for robotic operation to articulate the distal portion of the catheter sheathing.

Example 9—The articulating microwave ablation catheter of one of the preceding examples, wherein upon articulation of the microwave ablation catheter, the ablation zone of the radiating sections moves between 1 and 3 mm in a direction of the articulation.

Example 10—A method comprising: receiving an indication that a microwave ablation catheter has been navigated to location within the lungs of a patient; and articulating the microwave ablation catheter, wherein articulation adjusts an ablation zone of a radiating section of the microwave ablation catheter between 1 and 3 mm in a direction of the articulation.

Example 11—The method of example 10, wherein the articulation ensures that a therapy margin is achieved around a lesion.

Example 12—The method of examples 10 or 11, further comprising planning a navigation pathway through the lungs of the patient; and/or further comprising planning an articulation of the microwave ablation catheter.

Example 13—The method of example 12, wherein the navigation pathway is planned from pre-procedure images.

Example 14—The method of claims 10-13, further comprising: acquiring images to confirm placement of the microwave ablation catheter relative to a lesion in the patient; and/or acquiring images to assess a completeness of an ablation; and/or straightening or articulating the microwave ablation catheter in a second direction to alter the ablation zone to a second portion of the lesion.

Example 15—The method of example 14, further comprising imaging the lesion to confirm a margin around the lesion; and/or determining whether more lesions are identified in a pathway plan, receiving an indication that the microwave ablation catheter is at a location of a second lesion, articulating the microwave ablation catheter, and ablating the second lesion.

While detailed embodiments are disclosed herein, the disclosed embodiments are merely examples of the disclosure, which may be embodied in various forms and aspects. Therefore, specific structural and functional details disclosed herein are not to be interpreted as limiting, but merely as a basis for the claims.

We claim:

1. An articulating microwave ablation catheter comprising:
a catheter sheathing housing therein a radiating section defining an ablation zone;
a first pull wire traversing the catheter sheathing;
a pull ring secured in a distal portion of the catheter sheathing and configured to receive the pull wire; and
a drive mechanism operably connected to the catheter sheathing and the pull wire, wherein operation of the drive mechanism alters: an effective length of the pull wire, a shape of a distal portion of the catheter sheathing, and a location of the ablation zone defined by the radiating section.

2. The articulating microwave ablation catheter of claim 1, wherein the radiating section is a dipole antenna.

3. The articulating microwave ablation catheter of claim 1, further comprising a second pull wire, wherein the second pull wire and the first pull wire operate in concert, with one effectively lengthening and one effectively shortening, to enable articulation of the microwave ablation catheter in at least two directions.

4. The articulating microwave ablation catheter of claim 1, wherein the drive mechanism is manually operated and located in a handle connected to the catheter sheathing.

5. The articulating microwave ablation catheter of claim 1, wherein the catheter sheathing is configured to be received in a navigation catheter.

6. The articulating microwave ablation catheter of claim 5, wherein the radiating section is housed in a distal portion of the catheter sheathing, and the distal portion of the catheter sheathing is configured to extend beyond a distal end of the navigation catheter.

7. The articulating microwave ablation catheter of claim 1, wherein the pull wire is formed of graphite or carbon fiber.

8. The articulating microwave ablation catheter of claim 1, wherein the drive mechanism is configured for manual operation to articulate the distal portion of the catheter sheathing.

9. The articulating microwave ablation catheter of claim 1, wherein the drive mechanism is configured for robotic operation to articulate the distal portion of the catheter sheathing.

10. The articulating microwave ablation catheter of claim 1, wherein upon articulation of the microwave ablation catheter, the ablation zone of the radiating sections moves between 1 and 3 mm in a direction of the articulation.

11. A method comprising:
receiving an indication that a microwave ablation catheter has been navigated to location within the lungs of a patient; and
articulating the microwave ablation catheter, wherein articulation adjusts an ablation zone of a radiating section of the microwave ablation catheter between 1 and 3 mm in a direction of the articulation.

12. The method of claim 11, wherein the articulation ensures that a therapy margin is achieved around a lesion.

13. The method of claim 11, further comprising planning a navigation pathway through the lungs of the patient.

14. The method of claim 13, wherein the navigation pathway is planned from pre-procedure images.

15. The method of claim 13, further comprising planning an articulation of the microwave ablation catheter.

16. The method of claim 13, further comprising acquiring images to confirm placement of the microwave ablation catheter relative to a lesion in the patient.

17. The method of claim 16, further comprising acquiring images to assess a completeness of an ablation.

18. The method of claim 17, further comprising straightening or articulating the microwave ablation catheter in a second direction to alter the ablation zone to a second portion of the lesion.

19. The method of claim 18, further comprising imaging the lesion to confirm a margin around the lesion.

20. The method of claim 19, further comprising determining whether more lesions are identified in a pathway plan, receiving an indication that the microwave ablation catheter is at a location of a second lesion, articulating the microwave ablation catheter, and ablating the second lesion.
